(19) United States
(12) Patent Application Publication
(10) Pub. No.: US 2025/0090610 A1
(43) Pub. Date: Mar. 20, 2025

(54) COMPOSITION, AND EXTRACT OF CHLAMYDOMONAS REINHARDTII

(71) Applicant: HONDA MOTOR CO., LTD., Tokyo (JP)

(72) Inventors: Nozomi SHIOBARA, Saitama (JP); Shohei KINOSHITA, Saitama (JP); Mizuho DOI, Saitama (JP); Kenji MACHIDA, Saitama (JP); Minoru GOTO, Saitama (JP)

(21) Appl. No.: 18/291,577

(22) PCT Filed: Aug. 2, 2022
(86) PCT No.: PCT/JP2022/029680

§ 371 Date: Jan. 24, 2024

(30) Foreign Application Priority Data
Sep. 30, 2021 (JP) 2021-162268

(51) Int. Cl. A61K 36/05; C12N 1/12; C12R 1/89

(52) U.S. Cl. CPC A61K 36/05; C12N 1/125; C12R 2001/89

(57) ABSTRACT

Provided is a composition comprising Chlamydomonas reinhardtii or an extract of the Chlamydomonas reinhardtii, wherein the Chlamydomonas reinhardtii or the extract of the Chlamydomonas reinhardtii functions as an antagonist of an adrenergic receptor.

### TECHNICAL FIELD

The present invention relates to a composition including a green alga Chlamydomonas reinhardtii or an extract of a green alga Chlamydomonas reinhardtii and to an extract of a green alga Chlamydomonas reinhardtii.

### BACKGROUND ART

Adrenergic receptors, which are classified into nine types: α₁ (α_{1A}, α_{1B}, and α_{1D}), α2 (α_{2A}, α_{2B}, and α_{2C}), and β (β₁, β₂, and β₃), have a variety of actions. Thus, development of antagonists (antagonistic agents) of adrenergic receptors has been underway.

Meanwhile, a known example of the green alga Chlamydomonas reinhardtii (Chlamydomonas reinhardtii) is a Honda DREAMO strain (accession number FERM BP-22306) (see, for example, Patent Document 1).

### CITATION LIST

### Patent Document

Patent Document 1: PCT International Publication No. WO2017/217116

### DISCLOSURE OF THE INVENTION

### Problems to be Solved by the Invention

However, it is not known whether a green alga Chlamydomonas reinhardtii or an extract of a green alga Chlamydomonas reinhardtii can act as an antagonist of an adrenergic receptor.

It is an object of the present invention to provide a composition including a green alga Chlamydomonas reinhardtii or an extract of a green alga Chlamydomonas reinhardtii as an active ingredient that acts as an antagonist of an adrenergic receptor.

### Means for Solving the Problems

An aspect of the present invention is directed to a composition including a green alga Chlamydomonas reinhardtii or an extract of a green alga Chlamydomonas reinhardtii, in which the green alga Chlamydomonas reinhardtii or the extract of the green alga Chlamydomonas reinhardtii acts as an antagonist of an adrenergic receptor.

The green alga Chlamydomonas reinhardtii may be a UTEX 90 strain.

The green alga Chlamydomonas reinhardtii may be a Honda DREAMO strain (accession number FERM BP-22306).

The composition may be an antipsychotic drug, an antiglaucoma drug, a hypotensive drug, a therapeutic drug for angina pectoris, a peripheral vasodilator drug, an antiarrhythmic drug, a vasodilator drug, a dysuria improving drug, an antiemetic drug, or an antidepressant drug.

The adrenergic receptor may be an adrenergic receptor α_{1B}, an adrenergic receptor α_{2B}, or an adrenergic receptor β₂.

Another aspect of the present invention is directed to a product including an extract of a green alga Chlamydomonas reinhardtii, in which the extract acts as an antagonist of an adrenergic receptor.

The green alga Chlamydomonas reinhardtii may be a UTEX 90 strain.

The green alga Chlamydomonas reinhardtii may be a Honda DREAMO strain (accession number FERM BP-22306).

The extract of the green alga Chlamydomonas reinhardtii may be a dimethyl sulfoxide extract of the green alga Chlamydomonas reinhardtii.

### Effects of the Invention

The present invention provides a composition including a green alga Chlamydomonas reinhardtii or an extract of a green alga Chlamydomonas reinhardtii as an active ingredient that acts as an antagonist of an adrenergic receptor.

### PREFERRED MODE FOR CARRYING OUT THE INVENTION

Hereinafter, embodiments of the present invention will be described.

An embodiment of the present invention is directed to a composition including a green alga Chlamydomonas reinhardtii or an extract of a green alga Chlamydomonas reinhardtii, in which the green alga Chlamydomonas reinhardtii or the extract of the green alga Chlamydomonas reinhardtii acts as an antagonist of an adrenergic receptor.

The green alga Chlamydomonas reinhardtii is typically, but not limited to, a UTEX 90 strain or a Honda DREAMO strain (accession number FERM BP-22306). The UTEX 90 strain is the parent of the Honda DREAMO strain.

The adrenergic receptor may be an adrenergic receptor α_{1A}, an adrenergic receptor α_{1B}, an adrenergic receptor α_{1D}, an adrenergic receptor α_{2A}, an adrenergic receptor α_{2B}, an adrenergic receptor α_{2C}, an adrenergic receptor β₁, an adrenergic receptor β₂, or an adrenergic receptor β₃. In particular, the adrenergic receptor is preferably an adrenergic receptor α_{1B}, an adrenergic receptor α_{2B}, or an adrenergic receptor β₂.

The composition according to an embodiment of the present invention, which includes a green alga Chlamydomonas reinhardtii or an extract of a green alga Chlamydomonas reinhardtii as an active ingredient that acts as an antagonist of an adrenergic receptor, is suitable for use as an antipsychotic drug, an antiglaucoma drug, a hypotensive drug, a therapeutic drug for angina pectoris, a peripheral vasodilator drug, an antiarrhythmic drug, a vasodilator drug, a dysuria improving drug, an antiemetic drug, an antidepressant drug, or any other pharmaceutical.

Such a pharmaceutical may be in an oral dosage form (internal preparation) or a parenteral dosage form (external preparation or injection).

Examples of the dosage form of such a pharmaceutical include, but are not limited to, solid formulations, such as tablets, granules, powders, and capsules, liquid formulations, such as solutions, suspensions, and emulsions, and freeze-dried formulations.

Such a pharmaceutical may be produced using known methods.

Besides the pharmaceutical, the composition according to an embodiment of the present invention may be used as a quasi-drug, a cosmetic, a food product, or any other non-pharmaceutical product.

The composition according to an embodiment of the present invention is preferably for human use, while it may be for non-human animal use.

The green alga Chlamydomonas reinhardtii may be cultured by any suitable method, such as static culturing, shake culturing, submerged culturing, or aerated culturing.

The culturing of the green alga Chlamydomonas reinhardtii may be performed using any suitable medium, such as a TAP medium or a urea medium.

The culturing of the green alga Chlamydomonas reinhardtii may be performed at any suitable temperature, such as a temperature of 2° C. or more and 38° C. or less.

The culturing of the green alga Chlamydomonas reinhardtii may be performed using any suitable cycle of light and dark periods, such as a 24 hour cycle with a light period of 6 hours or more and 24 hours or less.

The light period may provide any suitable level of photosynthetically effective photon flux density, such as a photon flux density of 50 μmol/m²/s or more and 2,000 μmol/m²/s or less.

The cultured green alga Chlamydomonas reinhardtii may be collected by any suitable method, such as centrifugation or filtration.

The collected green alga Chlamydomonas reinhardtii may be dried if necessary.

The green alga Chlamydomonas reinhardtii may be dried by any suitable method, such as lyophilization.

The green alga Chlamydomonas reinhardtii may be subjected to extraction using any suitable method, such as ultrasonic extraction.

The extraction of the green alga Chlamydomonas reinhardtii may be performed using any suitable solvent, examples of which include lower alcohols, such as methanol, ethanol, propyl alcohol, and isopropyl alcohol; lower aliphatic ketones, such as acetone and methyl ethyl ketone; polyhydric alcohols, such as 1,3-butylene glycol, propylene glycol, and glycerol; and hydrophilic organic solvents, such as dimethyl sulfoxide (DMSO). In particular, the solvent is preferably DMSO.

The liquid extract obtained from the green alga Chlamydomonas reinhardtii may be collected by any suitable method, such as centrifugation or filtration.

The collected green alga Chlamydomonas reinhardtii extract may be dried if necessary.

The green alga Chlamydomonas reinhardtii extract may be dried by any suitable method, such as lyophilization.

### EXAMPLES

Hereinafter, the present invention will be described with reference to examples, which are not intended to limit the present invention.

### Example 1

### Culturing of Green Alga Chlamydomonas reinhardtii

The Honda DREAMO strain of the green alga Chlamydomonas reinhardtii was statically cultured for 5 days (seed culturing) using 2 L of a TAP medium under the conditions shown below.
Temperature: 25° C.
Light and dark cycles: 12 hour light period and 12 hour dark period
Photosynthetically effective photon flux density during light period: 60 to 80 μmol/m²/s
Light source: White LED
Subsequently, 500 mL of the resulting seed culture was centrifuged at 3,000 rpm for 5 minutes, and then the resulting culture supernatant was removed, so that the seed-cultured Honda DREAMO strain was collected.

Next, the collected Honda DREAMO strain was statically cultured for 7 days (preculturing) using 2 L of a TAP medium under the same conditions as those for the seed culturing.

Next, the precultured Honda DREAMO strain was cultured with aeration for 7 days (main culturing) using 5 L of a urea medium under the same conditions as those for the seed culturing.

Subsequently, 500 mL of the main culture was centrifuged at 6,000 rpm for 10 minutes, and the resulting culture supernatant was removed, so that the main-cultured Honda DREAMO strain was collected.

### Extraction

Using an ultrasonic homogenizer, approximately 20 mL of the collected Honda DREAMO strain was sonicated and extracted with 100 mL of DMSO for 5 minutes. The ultrasonic extraction was performed on ice.

Next, the extract and residue mixture was centrifuged at 3,000 rpm for 5 minutes, and then the residue was removed, while approximately 100 mL of the extract was collected.

The extract was then lyophilized to give 490.74 mg of a Honda DREAMO strain extract.

### Sample Preparation

Using an ultrasonic homogenizer, 490 mg of the Honda DREAMO strain extract was dissolved in 4.9 L of DMSO to form a DMSO solution of 0.1 g/L of the Honda DREAMO strain extract.

The DMSO solution of the Honda DREAMO strain extract was then centrifuged at 15,000 rpm for 5 seconds. The resulting supernatant was then collected to give a sample.

Next, two types of tests shown below were performed to determine at what concentration the sample was to be subjected to an assay (in vitro function evaluation assay).

### Cell Growth Inhibition Test

HEK293 cells (human embryonic kidney cell-derived cell line) were grown in a D-MEM (high glucose)+10% FBS+1% Penicillin-Streptomycin medium. To the resulting culture was added the sample at 13 different concentrations (0, 10, 20, 30, 40, 50, 60, 70, 80, 90, 100, 250, and 500 μg/mL). The resulting mixtures were subjected to a dose dependence test. Twenty-four hours after the addition of the sample to the cell culture, the medium was replaced with fresh one, and the cells were stained with WST-8 for 4 hours. The staining was followed by measuring the optical density (O.D.) at a wavelength of 450 nm using FlexStation 3 Multimode Microplate Reader (manufactured by Molecular Devices). In this case, the test was performed with n=3.

As a result, no decrease in optical density was observed at any of the sample concentrations, and therefore, it was determined that the concentrations had no influence on the cell growth.

### Test to Check the Influence of the Sample on Optical Density

To the D-MEM (high glucose)+10% FBS+1% Penicillin-Streptomycin medium was added the sample at 13 different concentrations (0, 10, 20, 30, 40, 50, 60, 70, 80, 90, 100, 250, and 500 μg/mL). Twenty-four hours after the addition of the sample, the medium was replaced with fresh one, which was followed by staining with WST-8 for 4 hours. The staining was followed by measuring the optical density at a wavelength of 450 nm using FlexStation 3 Multimode Microplate Reader (manufactured by Molecular Devices). The test was performed with n=3.

As a result, an increase in optical density was observed when the concentration of the sample was 250 μg/mL and 500 μg/mL. This indicated that adding the sample at a concentration of 250 μg/mL or 500 μg/mL affected the optical density regardless of the presence or absence of the cells. On the other hand, adding the sample at a concentration of 100 μg/mL or less was found to have no influence on the optical density.

On the basis of the results, the sample concentration for the assay was set to at most 100 μg/mL, which was found to have no influence on the cell growth inhibition or the optical density of the sample.

### Assay

The sample was subjected to an assay using adrenergic receptor α_{1B}, adrenergic receptor α_{2B}, and adrenergic receptor β₂ as target molecules. In the assay, the concentration of the sample was 100 μg/mL. The assay was performed two times (n=2). The assay was outsourced to Eurofins Pharma Discovery Services, which performed the assay using two types of assay systems to eliminate false positives. In this case, an inhibition rate of 50% or more was determined to indicate antagonistic action on the target molecule.

Table 1 shows the results of the assay performed on the sample.

TABLE 1

| | | Inhibition | Efficacy of drug |
|---|---|---|---|
| Target molecule | Action | rate [%] | targeting the molecule |
| Adrenergic | Antagonist | 62 | Hypotensive, therapeutic |
| receptor α _{1B} | | | for angina pectoris, |
| | | | peripheral vasodilator, |
| | | | antiarrhythmic, vasodilator, |
| | | | dysuria improving |
| Adrenergic | Antagonist | 60 | Antiemetic, antipsychotic, |
| receptor α _{2B} | | | antidepressant |
| Adrenergic | Antagonist | 75 | Antiglaucoma, antiarrhythmic, |
| receptor β ₂ | | | hypotensive, vasodilator |

Table 1 shows that the Honda DREAMO strain extract contained in the sample can act as an antagonist of adrenergic receptor α_{1B}, adrenergic receptor α_{2B}, and adrenergic receptor β₂.

### Example 2

The sample was subjected to an assay as in Example 1 except that the green alga Chlamydomonas reinhardtii used was the UTEX 90 strain.

Table 2 shows the results of the assay performed on the sample.

TABLE 2

| Target | | Inhibition | Efficacy of drug targeting |
|---|---|---|---|
| molecule | Action | rate [%] | the molecule |
| Adrenergic | Antagonist | 89 | Hypotensive, therapeutic |
| receptor α _{1B} | | | for angina pectoris, |
| | | | peripheral vasodilator, |
| | | | antiarrhythmic, vasodilator, |
| | | | dysuria improving |
| Adrenergic | Antagonist | 104 | Antiemetic, antipsychotic, |
| receptor α _{2B} | | | antidepressant |
| Adrenergic | Antagonist | 95 | Antiglaucoma, antiarrhythmic, |
| receptor β ₂ | | | hypotensive, vasodilator |

Table 2 shows that the UTEX 90 strain extract contained in the sample can act as an antagonist of adrenergic receptor α_{1B}, adrenergic receptor α_{2B}, and adrenergic receptor β₂.

1. A composition comprising a green alga Chlamydomonas reinhardtii or an extract of a green alga Chlamydomonas reinhardtii,
wherein the green alga Chlamydomonas reinhardtii or the extract of the green alga Chlamydomonas reinhardtii acts as an antagonist of an adrenergie receptor.

2. The composition according to claim 1, wherein the green alga Chlamydomonas reinhardtii is a UTEX 90 strain.

3. The composition according to claim 1, wherein the green alga Chlamydomonas reinhardtii is a Honda DREAMO strain (accession number FERM BP-22306).

4. The composition according to claim 1, wherein the composition is an antipsychotic drug, an antiglaucoma drug, a hypotensive drug, a therapeutic drug for angina pectoris, a peripheral vasodilator drug, an antiarrhythmic drug, a vasodilator drug, a dysuria improving drug, an antiemetic drug, or an antidepressant drug.

5. The composition according to claim 1, wherein the adrenergic receptor is an adrenergic receptor α_{1B}, an adrenergic receptor α_{2B}, or an adrenergic receptor β₂.

6. An extract of a green alga Chlamydomonas reinhardtii,
the extract acting as an antagonist of an adrenergic receptor.

7. The extract of a green alga Chlamydomonas reinhardtii according to claim 6, wherein the green alga Chlamydomonas reinhardtii is a UTEX 90 strain.

8. The extract of a green alga Chlamydomonas reinhardtii according to claim 6, wherein the green alga Chlamydomonas reinhardtii is a Honda DREAMO strain (accession number FERM BP-22306).

9. The extract of a green alga Chlamydomonas reinhardtii according to claim 6, wherein the extract of the green alga Chlamydomonas reinhardtii is a dimethyl sulfoxide extract of the green alga Chlamydomonas reinhardtii.
